(19) United States
(12) Patent Application Publication
(10) Pub. No.: US 2008/0294000 A1
(43) Pub. Date: Nov. 27, 2008

(54) ENDOSCOPE SUPPORT SYSTEM

(75) Inventor: Toru IWAMOTO, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/062,611

(22) Filed: Apr. 4, 2008

(30) Foreign Application Priority Data
Apr. 6, 2007 (JP) 2007-100047

(51) Int. Cl. A61B 1/00

(52) U.S. Cl. 600/103

(57) ABSTRACT

There is provided an endoscope support system including an endoscope device and a support device. The endoscope device includes an endoscope monitor, an electronic endoscope, an image processing unit to process an image picked up by the electronic endoscope to display the image on the endoscope monitor, and an image transmission unit to transmit image data corresponding to the image to the support device. The support device includes a support device monitor, a display control unit to display an image of the image data, a position input unit to accept an input pointing to a section in the image and to obtain position information, and a position transmission unit to transmit the position information to the endoscope device. Further, the endoscope device includes an image superimposing unit to superimpose a predetermined image at a point corresponding to the position information on the image displayed on the endoscope monitor.

### BACKGROUND OF THE INVENTION

The present invention relates to an endoscope support system for providing instructions to an operator of an endoscope device from a remote location.

Recently, endoscopic surgery has become widespread. However, the degree of penetration of specialized facilities where endoscopic surgery can be performed varies considerably from region to region. That is, the number of specialized facilities where endoscopic surgery can be performed and the number of surgeons specialized in endoscopic surgery are still limited. Therefore, if endoscopic surgery is required for a patient living in a region where the endoscopic surgery has not become widespread, it is necessary to dispatch the patient and a patient's doctor to a specialized facility of endoscopic surgery so that the endoscopic surgery can be performed by a specialized surgeon (specialized in endoscopic surgery) while at the same time being observed by the patient's doctor at the specialized facility. Such endoscope surgery involves not only the patient but also the patient's doctor for a relatively long time. Sometimes, the patient and the patient's doctor need to be involved in the endoscopic surgery for several days, which is uneconomical.

In view of the above mentioned circumstances, an endoscope support system where endoscopic surgery can be performed without the need for dispatching a patient's doctor to a specialized facility has been suggested in Japanese Patent Provisional Publication No. 2005-21354A (hereafter, referred to as JP2005-21354A). In the endoscope support system disclosed in JP2005-21354A, only a patient is dispatched to the specialized facility and the patient's doctor gives instructions, from a remote location, to a specialized surgeon at the specialized facility.

More specifically, in the endoscope support system disclosed in JP2005-21354A, a endoscope device to be operated by the specialized surgeon and a support device to be used by the patient's doctor are connected to each other via a network (e.g., Internet). An image picked up by an endoscope of the endoscope device is transmitted to and displayed on the support device. The patient's doctor is allowed to observe the image of the patient's body on a monitor of the support device and to write a marker or a message on the image displayed on the monitor of the support device. The image to which a marker or a message is added is then returned to the endoscope device of the specialized surgeon through the network. Then, the specialized surgeon performs the endoscopic surgery while checking the marker or the message on the image transmitted from the support device. In order to enable the patient's doctor to give instructions to the specialized surgeon appropriately, images of the patient picked up by the endoscope are transmitted to the support device of the patient's doctor as a moving image, and images to which the marker or the message is added by the patient's doctor are transmitted to the endoscope device as a moving image.

As described above, in the endoscope support system disclosed in JP2005-21354A, moving images are exchanged between the endoscope device and the support device. Therefore, the endoscope support system may put a heavy load on the traffic of the network. There may be a case where a relatively long time period is required for the endoscope device to receive a replay (i.e., an instruction) from the patient's doctor after transmitting an image to the support device of the patient's doctor. That is, there may be a case where the specialized surgeon is not able to receive instructions from the patient's doctor in real time.

In general, data of a moving image is compressed when the data of the moving image is to be transmitted through the network to reduce the data amount of the data of the moving image. Therefore, in the endoscope support system disclosed in JP2005-21354A, the support device of the patient's doctor is also required to compress (i.e., to encode) data of the image to which the marker or the message is added. That is, the above mentioned endoscope support system requires the support device of the patient's doctor to have an expensive real-time encoder having high performance. It should be noted that the support device of the patient's doctor is rarely used for the remote endoscope surgery.

In addition, the above mentioned endoscope support system requires each of the endoscope device and the support device to use an internet connection service supporting a high data transmitting speed as well as supporting a high data receiving speed. That is, in addition to the endoscope device in the specialized facility, the support device on the patient's doctor side is required not to use an asymmetric connection service, such as an ADSL (Asymmetric Digital Subscriber Line), but a symmetric high speed internet connection service, such as an FTTH (Fiber To The Home), which is expensive. In summary, the above mentioned endoscope support system puts a heavy load on the support device of the patient's doctor.

### SUMMARY OF THE INVENTION

The present invention is advantageous in that it provides an endoscope support system capable of reducing a load imposed on a support device of a patient's doctor.

According to an aspect of the invention, there is provided an endoscope support system including an endoscope device, and a support device connected to the endoscope device via a network. In this configuration, the endoscope device is provided with an endoscope monitor having a screen, an electronic endoscope, an image processing unit configured to process an image picked up by the electronic endoscope to display the image on the endoscope monitor, and an image transmission unit configured to transmit image data corresponding to the image to the support device via the network. The support device is provided with a support device monitor having a screen, a display control unit configured to display, on the support device monitor, an image corresponding to the image data received from the endoscope device, a position input unit configured to accept an input pointing to a section in the image displayed on the support device monitor and to obtain position information corresponding to the section in the image displayed on the support device monitor, and a position transmission unit configured to transmit the position information obtained by the position input unit to the endoscope device. Further, the endoscope device includes an image superimposing unit configured to superimpose a predetermined image at a point corresponding to the position information transmitted from the position transmission unit of the support device on the image displayed on the endoscope monitor.

The above mentioned configuration makes it possible to display a predetermined image, such as a marker, at a desired point in the image displayed on the endoscope monitor by operating the support device. Since the support device only needs to display the image transmitted from the endoscope device, to input position information of a desired section in the displayed image, and to transmit the position information to the endoscope device, a general-purpose personal computer can be used for the support device. There is no necessity to prepare a dedicated device for the support device. Data transmitted from the support device to the endoscope device is the position information whose data amount is small. Therefore, the support device is allowed to use a low-cost network connection service (e.g., ADSL) providing a relatively low data transmission speed.

In at least one aspect, the position input unit includes a pointing device operated to point to the section and to input a coordinate of the section in the image displayed on the support device monitor.

In at least one aspect, the support device further includes a text information input unit configured to accept an input of text information, and a text information transmission unit configured to transmit the text information accepted by the text information input unit to the endoscope device. Further, the endoscope device includes a text superimposing unit configured to superimpose the text information transmitted from the text information transmission unit of the support device on the image displayed on the endoscope monitor.

In at least one aspect, the predetermined image includes a marker.

In at least one aspect, the marker includes an arrow.

In at least one aspect, the network includes an Internet.

In at least one aspect, the support device includes a marker type input unit configured to accept an input designating a type of a marker to be superimposed, and a marker type transmission unit configured to transmit marker type information indicating the type of the marker designated by the marker type input unit to the endoscope device. In this configuration, the image superimposing unit of the endoscope device superimposes, as the predetermined image, a marker corresponding to the marker type information on the image on the endoscope monitor.

### BRIEF DESCRIPTION OF THE ACCOMPANYING DRAWINGS

FIG. 1 illustrates a system configuration of an endoscope support system according to an embodiment of the invention.

FIG. 2 is a block diagram of an endoscope device provided in the endoscope support system.

FIG. 3 illustrates a block diagram of a support device provided in the endoscope support system.

FIG. 4 illustrates an application window displayed on a support device monitor of the support device.

### DETAILED DESCRIPTION OF THE EMBODIMENTS

Hereinafter, an embodiment according to the invention are described with reference to the accompanying drawings.

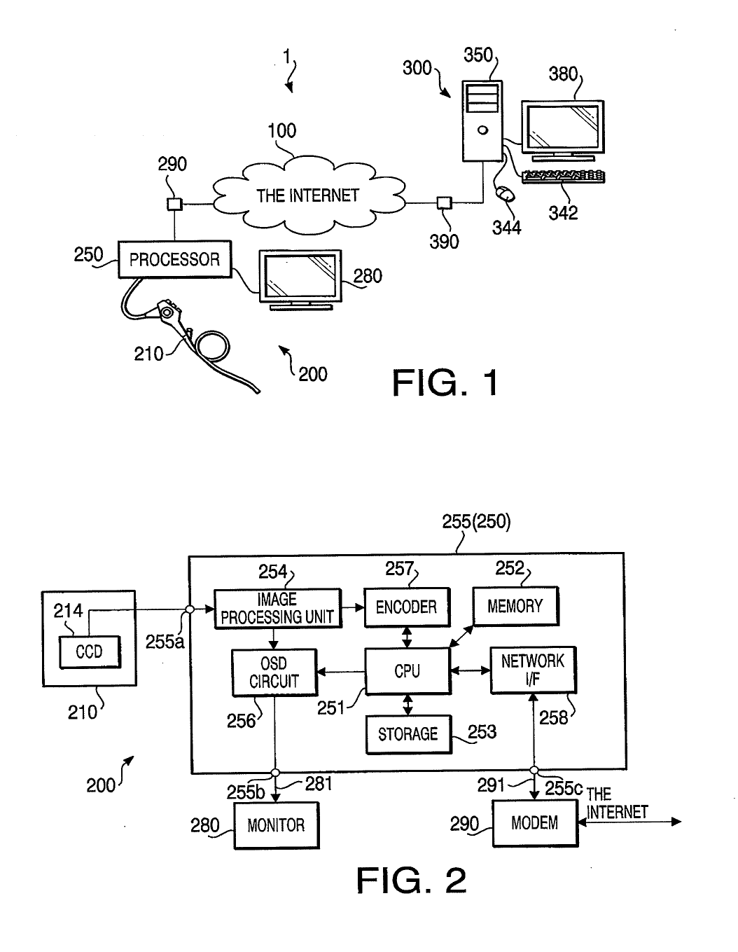

FIG. 1 illustrates a system configuration of an endoscope support system 1 according to an embodiment of the invention. In the endoscope support system 1, an endoscope device 200 and a support device 300 are connected to each other via the Internet 100.

The endoscope device 200 includes an electronic endoscope 210, a processor 250, an endoscope monitor 280 and a modem 290. The electronic endoscope 210 has an image pick-up device (e.g., CCD) 214 at a distal end portion thereof to pick up an image of an object in the vicinity of the distal end portion (see FIG. 2). The image picked up by the CCD 214 is transmitted to the processor 250 as an image signal. The processor 250 processes the image signal transmitted from the electronic endoscope 210 to generate a video signal having a predetermined signal format (e.g., an NTSC signal). The video signal generated by the processor 250 is transmitted to the endoscope monitor 280. Consequently, an image picked up by the electronic endoscope 210 is displayed on the endoscope monitor 280.

The CCD 214 is controlled to pick up images repeatedly at predetermined time intervals of, for example, 30 seconds, and to transmit the picked up images to the processor 250. Therefore, the images picked up by the electronic endoscope 210 are displayed on the endoscope monitor 280 as a moving image.

To the processor 250, the modem 290 is connected. Therefore, the processor 250 is able to transmit data to and receive data from an external host device (e.g., the support device 300) via the Internet 100. The modem 290 has the function of transmitting and receiving data at a relatively high speed (e.g., several tens of millions of bits/s). The modem 290 is, for example, an FTTH modem.

The support device 300 includes a workstation 350, a keyboard 342, a mouse 344, a support device monitor 380 and a modem 390. On the workstation 350, a general-purpose OS (e.g., Microsoft Windows®) has been installed. By executing an application program on the OS of the workstation 350, a function of supporting an operator of the endoscope device 200 is implemented on the support device 300.

To the workstation 350, the keyboard 342, the mouse 344 and the support device monitor 380 are connected. A user of the workstation 350 is able to input text information to the workstation 350 through the keyboard 342 and to input a coordinate to the workstation 350 by operation the mouse 344. The output of the application program running on the workstation 350 is displayed on the support device monitor 380.

To the workstation 350, the modem 390 is connected. Therefore, the workstation 350 is able to transmit data to and receive data from an external host device (e.g., the endoscope device 200) via the Internet 100. The modem 390 has the function of receiving data at a relatively high speed (e.g., several millions of bits/s through several tens of millions of bits/s). On the other hand, the modem 390 is allowed to have a relatively low the data transmitting speed (e.g., several hundred thousands of bits/s through several millions of bits/s). The modem 390 is, for example, an ADSL modem.

Hereafter, the endoscope device 200 is explained in detail. FIG. 2 is a block diagram of the endoscope device 200. On a body case 255 of the processor 250, a connector 255a is provided so that the electronic endoscope 210 and the processor 250 are connected to each other via the connector 255a. By thus connecting the electronic endoscope 210 to the processor 250, the image signal output by the CCD 214 of the electronic endoscope 210 can be transmitted to the processor 250.

The processor 250 includes, in the body case 255, a CPU 251, a memory 252, a storage device 253, an image processing unit 254, an OSD circuit 256, an encoder 257 and a network interface 258. The network interface 258 interfaces the processor 250 with the network via a network port 255c provided on the body case 255. The network port 255c is, for example, an Ethernet® port. To the network port 255c, the modem 290 is connected via a network cable 291. In this configuration, the CPU 251 is able to transmit data to an external host device (e.g. the support device 300) and to receive data from the external host device via the modem 290 by controlling the network interface 258.

The image processing unit 254 samples the image signal transmitted from the CCD 214 to generate digital image data and executes a predetermined image process (e.g., an adjusting process for brightness, contrast and/or color) on the image data to convert the image data into a video signal having a predetermined signal format. The converted video signal is then transmitted to the OSD circuit 256.

The OSD circuit 256 has the function of superimposing a predetermined image which represents at least a symbol or a text character on the image corresponding to the video signal transmitted from the image processing unit 254 and transmitting the processed video signal to a monitor connector 255b provided on the body case 255. The endoscope monitor 280 is connected to the monitor connector 255b via a monitor cable 281. In this configuration, an endoscopic image on which an image (e.g., a symbol or a text character) is superimposed by the OSD circuit 256 is displayed on the endoscope monitor 280.

Images to be superimposed on the image corresponding to the video signal from the image processing unit 254 are provided from the CPU 251 to the OSD circuit 256. As described in detail later, the CPU 251 generates an image to be superimposed on the endoscopic image based on data received from the support device 300. As described later, the support device 300 is configured to allow the user of the support device 300 to input instructions concerning images to be superimposed.

The encoder 257 reads periodically the digital image data generated by the image processing unit 254 to obtain a plurality of pieces of image data arranged in time series and to generate a stream of moving image data having a predetermined data format (e.g., MPEG2 image data) based on the plurality of pieces of time series image data. After the stream of moving image data is transmitted to the CPU 251 once, the CPU 251 transmits the stream of moving image data to the support device 300 via the network interface 258. For example, an RTP (Real-time Transport Protocol) is used to transmit the stream of moving image data from the processor 250 to the support device 300.

When the encoder 257 generates the stream of moving image data, the encoder 257 compresses the image data from the image processing unit 25, for example, by a spectrum conversion scheme (e.g., a discrete cosine conversion or a wavelet conversion) to reduce a high frequency component concerning a gray scale of the image. Therefore, the encoder 257 is formed of, for example, a DSP (Digital Signal Processor) so that the images picked up by the electronic endoscope 210 can be converted to a moving image and the moving image can be transmitted to the support device 300 approximately in real time.

The memory 252 is a work memory for the CPU 251. The storage device 253 is, for example, an EEPROM, in which programs and data to achieve the above mentioned functions of the endoscope device 250 are stored.

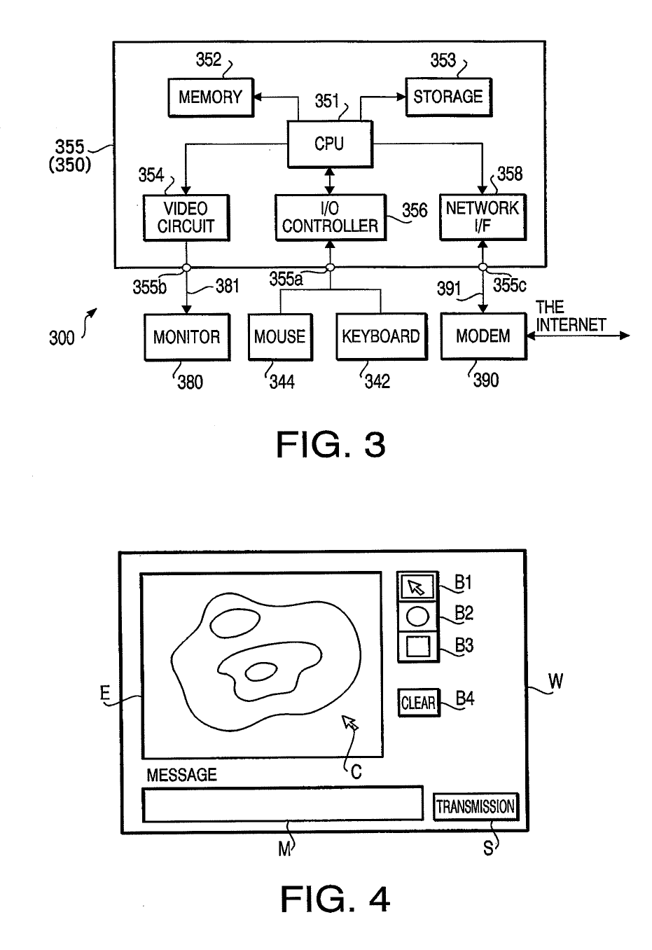

Hereafter, the support device 300 is explained in detail. FIG. 3 illustrates a block diagram of the support device 300. The workstation 350 includes, in a body case 355, a CPU 351, a memory 352, a storage device 353, a video circuit 354, an I/O controller 356, and a network interface 358. On the outer surface of the body case 355, an I/O port 355a, a monitor connector 355b, and a network port 355c are provided. To the I/O port 355a, the keyboard 342 and the mouse 344 are connected. To the monitor connector 355b, the support device monitor 380 is connected via a monitor cable 381. To the network port 355c, the modem 390 is connected via a network cable 391.

The CPU 351 controls the network interface 358 to transmit data to and to receive data from an external host via the network port 355c. As described above, the modem 390 is connected to the network port 355c. Therefore, the CPU 351 is able to communicate with the endoscope device 200 via the Internet 100.

As described above, a stream of moving image data is transmitted from the endoscope device 200 to the support device 300 via the Internet 100. The stream of moving image data is decoded by the CPU 351, and is transmitted to the video circuit 354. The video circuit 354 processes the decoded data to display the moving image on a certain area on the support device monitor 380. The moving image picked up by the electronic endoscope 210 is thus displayed on the support device monitor 380.

The CPU 351 controls the network interface 358 to transmit information concerning an image to be superimposed on the endoscopic image displayed on the endoscope monitor 280, to the endoscope device 200 via the Internet 100.

The I/O controller 356 may be a general-purpose I/O controller, such as a USB controller. The I/O controller 356 receives information, such as text information, inputted through the keyboard 342 connected to the I/O port 355a or a coordinate inputted by operating the mouse 344. The I/O controller 356 transmits the received information to the CPU 351. As described later, the user of the support device 300 is able to input and modify an image to be superimposed on the endoscopic image by operating the keyboard 342 and the mouse 344.

The memory 352 is used as a work memory for the CPU 352. In the storage device 353, a general-purpose OS (Operating System) of the workstation 350, application programs and data to be used by the OS and application programs are stored. The storage device 353 is, for example, an HDD.

As described above, the workstation 350 may be a general-purpose PC (Personal Computer) on which a general-purpose OS runs. By executing a support application program on the general-purpose OS running on the workstation 350, the user of the workstation 350 is allowed to give instructions to the endoscope device 200.

When the support application program is started, an application window W shown in FIG. 4 is displayed on the support device monitor 380. As shown in FIG. 4, in the window W, an endoscopic image display area E, tool selection buttons B1, B2 and B3, a clear button B4, a message input area M and a transmission button S are located.

In the endoscopic image display area E, an image picked up by the electronic endoscope 210 is displayed. That is, the stream of moving image data transmitted from the endoscope device 200 is decoded by the CPU 351 and the moving image corresponding to the decoded moving image data is displayed in the endoscopic image display area E.

The tool selection buttons B1-B3 are used to designate which of the types of the images (i.e., markers) should be superimposed on the endoscopic image. The user of the workstation 350 operates the mouse 344 to move a cursor C on a desired one of the tool selection buttons B1-B3, and then clicks a button of the mouse 344 to designate the type of the image to be superimposed on the endoscopic image. For example, the buttons B1, B2 and B3 respectively correspond to an arrow, a circle and a rectangle (see FIG. 4).

After clicking a desired one of the tool selection buttons B1-B3, the user moves the cursor C to a desired point on the endoscopic image displayed in the endoscopic image display area E. Then, the user operates the mouse 344 to determine a coordinate at which the selected marker is superimposed on the endoscopic image. For example, when the tool selection button B1 is selected (i.e., an arrow is used as a marker), the user moves the cursor C to a desired point on the endoscopic image, and then clicks the button of the mouse 344. Then, the CPU 351 obtains a relative coordinate of the cursor C with respect to an origin point defined at the upper-left corner of the endoscopic image display area E, and a selection result of tool selection buttons. In the above mentioned example, the selection result indicates that that the button B1 is selected. Then, the CPU 351 transmits information concerning the relative coordinate of the cursor C and the selection result of tool selection buttons to the processor 250 of the endoscope device 200 via the Internet 100.

The CPU 251 of the processor 250, which received the information concerning the relative coordinate of the cursor C and the selection result of tool selection buttons from the support device 300, controls the OSD circuit 256 to superimpose the selected marker (e.g., the arrow) at a point corresponding to the received relative coordinate in the endoscopic image displayed on the endoscope monitor 280. At this time, the selected marker (e.g., the arrow) is also displayed in the endoscopic image display area E of the support device 300. Therefore, the user of the support device 300 is also able to visually recognize the point where the selected marker is displayed.

The above mentioned process for superimposing the selected marker (button B1) also applies to the case where the tool selection button B2 or B3 is selected. That is, when the tool selection button B2 is selected, a circle is superimposed at a desired point on the endoscopic image of the endoscope monitor 280. On the other hand, when the tool selection button B3 is selected, a rectangular is superimposed at a desired point on the endoscopic image of the endoscope monitor 280. Namely, one of the three symbols to be displayed on the both monitors 280 and 380 as the marker can be selected.

Considering that endoscopic surgery is performed on the side of the endoscope device 200 and a patient's doctor is on the side of the support device 300, if the patient's doctor wants to conduct treatment (e.g., insertion of an endoscope to a body cavity, or blockage of a blood vessel by forceps) for a particular section of a patient's body displayed as the endoscopic image, the patient's doctor is able to superimpose a marker (e.g., an arrow) at the particular section in the endoscopic image. Consequently, a surgeon performing the endoscopic surgery is able to judge which section should be targeted for the treatment based on the point of the superimposed marker on the endoscope monitor 280.

The user of the support device 300 is also able to input text information to the workstation 350 through the keyboard 342. The inputted text information is displayed in the message input area M of the support device monitor 380. When the transmission button S is clicked in a state where the text information is displayed in the message input area M, the text information displayed in the message input area M is transmitted to the processor 250 of the endoscope device 200.

The CPU 251 of the processor 250 which received the text information controls the OSD circuit 256 to superimpose the text information on the endoscope monitor 280. Thus, the user of the support device 300 is able to transmit a desired message to the side of the endoscope device 200.

If the user of the support device 300 clicks the clear button B4 after finishing the above mentioned operation for designating the marker and inputting the text information, a clear command for clearing the superimposed image is transmitted to the processor 250 of the endoscope device 200 via the Internet 100. The CPU 251 of the processor 250 which received the clear command controls the OSD circuit 256 to clear the images (i.e., the marker and the text information) superimposed on the endoscopic image. Consequently, the marker and the message are removed from the endoscopic image, so that only the endoscopic image is displayed on the endoscope monitor 280.

As described above, according to the embodiment, it is possible to superimpose a desired image or a desired message on an endoscopic image of the endoscope monitor 280 to give instructions to the side of the endoscope device 200 from a remote location. In this regard, data transmitted from the support device 300 to the endoscope device 200 contains only a coordinate designated by the mouse, the type of the selected tool button and text information. Therefore, the amount of data transmitted from the support device 300 to the endoscope device 200 is small.

Such a configuration enables the support device 300 to use an internet connection service, such as an ADSL, which provides a relatively high data receiving speed and a relatively low data transmitting speed. In the communication between the endoscope device 200 and the support device 300, a predetermined protocol (e.g., a HTML) is used.

Although the present invention has been described in considerable detail with reference to certain preferred embodiments thereof, other embodiments are possible.

In the above mentioned embodiment, the type of the marker to be superimposed on the endoscope monitor 280 is set on the side of the support device 300. However, the type of the marker to be displayed may be determined on the side of the endoscope device 200. In this case, the application window W of FIG. 4 is configured not to have the tool selection buttons B1-B3 so that only a coordinate of a clicked point of the cursor is transmitted to the endoscope device 200 when the user moves the cursor at a desired point and clicks the mouse.

This application claims priority of Japanese Patent Application No. P2007-100047, filed on Apr. 6, 2007. The entire subject matter of the applications is incorporated herein by reference.

What is claimed is:

1. An endoscope support system, comprising:
an endoscope device; and
a support device connected to the endoscope device via a network,
wherein the endoscope device comprises:
an endoscope monitor having a screen;
an electronic endoscope;
an image processing unit configured to process an image picked up by the electronic endoscope to display the image on the endoscope monitor; and
an image transmission unit configured to transmit image data corresponding to the image to the support device via the network,
wherein the support device comprises:
a support device monitor having a screen;
a display control unit configured to display, on the support device monitor, an image corresponding to the image data received from the endoscope device;
a position input unit configured to accept an input pointing to a section in the image displayed on the support device monitor and to obtain position information corresponding to the section in the image displayed on the support device monitor; and
a position transmission unit configured to transmit the position information obtained by the position input unit to the endoscope device,
wherein the endoscope device further comprises an image superimposing unit configured to superimpose a predetermined image at a point corresponding to the position information transmitted from the position transmission unit of the support device on the image displayed on the endoscope monitor.

2. The endoscope support system according to claim 1, wherein the position input unit includes a pointing device operated to point to the section and to input a coordinate of the section in the image displayed on the support device monitor.

3. The endoscope support system according to claim 1,
wherein the support device further comprises:
a text information input unit configured to accept an input of text information; and
a text information transmission unit configured to transmit the text information accepted by the text information input unit to the endoscope device,
wherein the endoscope device comprises a text superimposing unit configured to superimpose the text information transmitted from the text information transmission unit of the support device on the image displayed on the endoscope monitor.

4. The endoscope support system according to claim 1, wherein the predetermined image includes a marker.

5. The endoscope support system according to claim 4, wherein the marker includes an arrow.

6. The endoscope support system according to claim 1, wherein the network includes an Internet.

7. The endoscope support system according to claim 1,
wherein the support device further comprises:
a marker type input unit configured to accept an input designating a type of a marker to be superimposed; and
a marker type transmission unit configured to transmit marker type information indicating the type of the marker designated by the marker type input unit to the endoscope device,
wherein the image superimposing unit of the endoscope device superimposes, as the predetermined image, a marker corresponding to the marker type information on the image on the endoscope monitor.
